(19) United States
(12) Patent Application Publication
(10) Pub. No.: US 2014/0349944 A1
(43) Pub. Date: Nov. 27, 2014

(54) CHAPERONE-BASED INTEGRIN INHIBITORS FOR THE TREATMENT OF CANCER AND INFLAMMATORY DISEASES

(71) Applicant: MUSC FOUNDATION FOR RESEARCH DEVELOPMENT, Charleston, SC (US)

(72) Inventors: Zihai LI, Charleston, SC (US); Feng HONG, Summerville, SC (US)

(73) Assignee: MUSC FOUNDATION FOR RESEARCH DEVELOPMENT, Charleston, SC (US)

(21) Appl. No.: 14/285,992

(22) Filed: May 23, 2014

Related U.S. Application Data
(60) Provisional application No. 61/826,654, filed on May 23, 2013.

(51) Int. Cl. C07K 14/705; A61K 47/48; A61K 38/17

(52) U.S. Cl. CPC C07K 14/70546; A61K 38/1777; A61K 47/48315
USPC 514/19.3; 530/327; 530/391.7; 514/21.5; 530/325

(57) ABSTRACT

The present disclosure provides isolated integrin αL polypeptides, such as α7 helix polypeptides from the alpha I domain of integrin. Such polypeptides inhibit the interaction between integrin and gp96, thereby inhibiting gp96 activity. Such inhibition can be used to prevent cancer cell growth, cancer metastasis and/or inflammation.

This application claims the benefit of U.S. Provisional Patent Application No. 61/826,654, filed May 23, 2013, the entirety of which is incorporated herein by reference.

The invention was made with government support under Grant Nos. AI070603 and AI077283 awarded by the National Institutes of Health. The government has certain rights in the invention.

### INCORPORATION OF SEQUENCE LISTING

The sequence listing that is contained in the file named “MESC.P0076US_ST25.txt”, which is 13 KB (as measured in Microsoft Windows®) and was created on May 23, 2014, is filed herewith by electronic submission and is incorporated by reference herein.

### BACKGROUND OF THE INVENTION

1. Field of the Invention

The present invention relates generally to the fields of medicine and cancer biology. More particularly, it concerns the development of novel integrin inhibitors to treat cancer metastasis, sepsis and autoimmune diseases.

2. Description of Related Art

Integrins are a large family of cell surface type I transmembrane receptors that mediate adhesion to the extracellular matrix and immunoglobulin superfamily molecules. At least 24 integrin heterodimers are formed by the combination of 18 α-subunits and 8 β-subunits (Barczyk et al., 2010). A wide variety of integrins have been shown to promote cancer cell proliferation, invasion and survival. For example, in melanoma, the αV subunit has been found to be strongly expressed in both benign and malignant lesions, whereas the β3 subunit is exclusively expressed in vertical growth stage and metastatic disease (Albelda et al., 1990; Natali et al., 1997). In addition, increased expression of the integrin α6β4 stimulates the survival of breast cancer cells (Weaver et al., 2002; Guo et al., 2006), and elevated expression of integrin α5β1 correlates with decreased survival in patients with lymph node-negative non-small-cell lung carcinoma (Dingemans et al., 2010). Moreover, integrin αL is up-regulated in CD44 stimulation-induced adhesion of colon cancer cells (Fujisaki et al., 1999), and integrin αL, αX, β1, β2 and ICAM are highly expressed in marginal zone B-cell lymphoma (Vincent et al., 1996; Matos et al., 2006). Furthermore, integrins on cancer stem cells have also been reported to play essential roles for cancer initiation and progression (Pontier et al., 2009). In recent years, novel insights into the mechanisms that regulate tumor progression have led to the development of integrin-based therapeutics for cancer treatment. Integrin inhibitors, including antibodies, peptides, and nonpeptidic molecules, are considered to have direct and indirect antitumor effects by restricting tumor growth and blocking angiogenesis. Several inhibitors have shown promise in preclinical studies and phase I and phase II trials, but phase III trials have reached no clinically significant results (Bolli et al., 2009; Makrilia et al., 2009; Desgrosellier et al., 2010). Vitaxin, a specific monoclonal antibody that targets the αvβ3 integrin, has shown significant antiangiogenic effects in preclinical studies and phase I/II trials (Brooks et al., 1994; Gutheil et al., 2000; McNeel et al., 2005). However, phase III trials have thus far shown no significant clinical benefits. Cligenitide is an RGD-based peptide which antagonizes αVβ3 integrins and has been administered to patients with cancers of the breast, lung, head and neck, but the results of those trials were not sufficiently encouraging to indicate further use in clinical practice (Burkhart et al., 2004; Raguse et al., 2004). Thus, there is a need for novel integrin inhibitors that could be employed as therapeutics, such as for cancer therapy. In addition, integrin also plays critical roles in leukocyte adhesion and activation. Blocking integrin is also expected to be beneficial for the treatment of sepis and autoimmune diseases (Vanderslice et al., 2006; and Cox et al., 2010).

### SUMMARY OF THE INVENTION

In first embodiment there is provided an isolated polypeptide comprising an α7 helix peptide domain of integrin (or a sequence having 1 or 2 amino acid substitutions, deletions or insertions relative to the α7 helix peptide domain), wherein the polypeptide is not a full length integrin polypeptide. For example, the α7 helix peptide domain from can be from integrin αL, such as human integrin αL (see, e.g., NCBI accession no. NP_{—}001107852 (SEQ ID NO: 11), incorporated herein by reference). In further aspects, the α7 helix peptide domain is from integrin αM or α4. In certain aspects, the α7 helix peptide domain comprises the sequence of SEQ ID NO: 1 or SEQ ID NO: 12 or a sequence having 1 or 2 amino acid substitutions, deletions or insertions relative to these sequences and is conjugated or fused to cell-targeting or a cell internalization moiety.

In certain embodiments, the invention provides an isolated polypeptide comprising an amino acid sequence of EKLKDLFTDLQR (SEQ ID NO: 1), EKLKDLFTELQK (SEQ ID NO: 12) or a sequence having 1 or 2 amino acid substitutions, deletions or insertions relative to SEQ ID NO: 1 or SEQ ID NO: 12. In certain aspects an isolated polypeptide comprises a sequence of SEQ ID NO: 1 or SEQ ID NO: 12, or a sequence that is at least 90% identical thereto. For example, in some aspects, the polypeptide comprises an amino acid sequence according to SEQ ID NO: 1 or SEQ ID NO: 12 or a sequence having 1 or 2 amino acid substitutions, deletions or insertions relative to SEQ ID NO: 1 or SEQ ID NO: 12, wherein the polypeptide is not a full-length integrin αL polypeptide. In some aspects, the isolated polypeptide is less than about 200, 150, 100, 90, 80, 70, 60, 50, 40 or 30 amino acids in length (or comprises less than about 200, 150, 100, 90, 80, 70, 60, 50, 40 or 30 contiguous amino acids amino acids of integrin αL). In still further aspects, a polypeptide can comprise a sequence that is about 90, 92, 94, 95, 96, 98, or 100% identical to SEQ ID NO:1 or SEQ ID NO: 12.

Furthermore it will be understood by the skilled artisan that an isolated polypeptide may comprise amino acid substitutions relative to SEQ ID NO: 1 or SEQ ID NO: 12. In some very specific aspects the isolated polypeptide may be identical to the sequence given by SEQ ID NO: 1 or SEQ ID NO: 12 (an integrin αL α7 helix sequence). In still further aspects, a polypeptide of the embodiments, comprises one or more amino acid position that is substituted with a non-natural amino acid. In yet further aspects, the polypeptide is defined a stabilized alpha helix polypeptide or a cyclic peptide.

In some further aspects an isolated polypeptide may comprise a cell internalization moiety. In some cases a cell internalization moiety may be conjugated to the isolated polypeptide. For example, the isolated polypeptide may be provided in complex with a liposomal vesicle thereby enabling the polypeptide to traverse the cell membrane. Furthermore, in some specific aspects, a cell internalization moiety may be a polypeptide, a polypeptide, an aptamer or an avimer (see for example U.S. Applns. 20060234299 and 20060223114, incorporated herein by reference) sequence. For example, a cell internalization moiety may comprise amino acids from the HIV TAT, HSV-1 tegument protein VP22, or Drosophila antennopedia homeodomain. In certain further aspects, a cell internalization moiety may be an engineered internalization moiety such as a poly-Arginine, a poly-methionine and/or a poly-glycine polypeptide such as Methionine and Glycine polypeptides. For example, a cell internalization moiety may be comprise a cell internalization moiety derived from the HIV tat protein, such a segment comprising the sequence GRKKRRQRRR (SEQ ID NO: 2) or YGRKKRRQRRR (SEQ ID NO: 4). Additional cell internalization moieties that may be used according to the embodiments include, with limitation the sequence of RMRRMRRMRR (SEQ ID NO: 5) or GRKKRRQRRRPQ (SEQ ID NO: 6). In some aspects, such cell internalization moieties may be fused to the N- or C-terminus of a polypeptide of the embodiments.

Thus, in some cases a polypeptide cell internalization moiety and the isolated polypeptide may form a fusion protein. The skilled artisan will understand that such fusion proteins may additionally comprises one or more amino acid sequences separating the cell internalizing moiety and the isolated polypeptide sequence. For example, in some cases a linker sequence may separate these two domains. For example, a linker sequences may comprise a “flexible” amino acids with a large number or degrees of conformational freedom such as a poly glycine linker. In some cases, a linker sequence may comprise a proteinase cleavage site. For instance, a linker sequence may comprise a cleavage site that is recognized and cleaved by an intracellular proteinase, thereby releasing the isolated polypeptide sequence from the cell internalization sequence once the fusion protein has been internalized.

In further aspects of the embodiments a polypeptide may comprise a cell targeting moiety, which is a moiety that binds to and/or is internalized by only a selected population of cells such as cells expressing a particular cellular receptor. Such a cell targeting may, for example, comprise an antibody, a growth factor, a hormone, a cytokine, an aptamer or an avimer that binds to a cell surface protein. As used herein the term antibody may refer to an IgA, IgM, IgE, IgG, a Fab, a F(ab′)2, single chain antibody or paratope polypeptide. In certain cases, a cell targeting moiety of the invention may target a particular type of cells such as a liver, skin, kidney, blood, retinal, endothelial, iris or neuronal cell. In still further aspects a cell targeting moiety of the invention may be defined as cancer cell binding moiety. For example, in some very specific cases a cell targeting moiety of the invention may target a cancer cell associated antigen such a gp240 or Her-2/neu.

In still further aspects of the embodiments the isolated polypeptide may comprise additional amino acid sequences such as a cell trafficking signal (e.g., a cell secretion signal, a nuclear localization signal or a nuclear export signal) or a reporter polypeptide such as an enzyme or a fluorescence protein. In a preferred aspect for example, the isolated polypeptide comprises a cellular secretion signal. Thus, in certain cases, the isolated polypeptide may comprise a cell internalization moiety and cell secretion signal, thereby allowing the polypeptide to be secreted by one cells and internalized into a surrounding a cell.

In a further embodiment, the invention provides an isolated polypeptide that comprises SEQ ID NO: 3 (GRKKRRQRRRPQEKLKDLFTDLQR) or SEQ ID NO: 13 (GRKKRRQRRRPQEKLKDLFTELQK), or a sequence that is at least 90% identical thereto. For example, in some aspects, the polypeptide comprises an amino acid sequence at least 90% identical to SEQ ID NO: 3, wherein the polypeptide is not a full-length integrin αL polypeptide. In some aspects, the isolated polypeptide is less than about 200, 150, 100, 90, 80, 70, 60, 50, 40 or 30 amino acids in length (or comprises less than about 200, 150, 100, 90, 80, 70, 60, 50, 40 or 30 contiguous amino acids amino acids of integrin αL). In still further aspects, a polypeptide can comprise a sequence that is about 90, 92, 94, 95, 96, 98, or 100% identical to SEQ ID NO: 3 or SEQ ID NO: 13. In certain cases, the isolated polypeptide may comprise an amino acid substitution, insertion or deletion of 1, 2, 3, 4, or 5 amino acids from SEQ ID NO: 3 or SEQ ID NO: 13. For example, in some aspects, an isolated polypeptide is providing comprising a polypeptide fragment of SEQ ID NO: 3 or SEQ ID NO: 13, having no more than 1, 2 or 3 amino acid substitutions, insertions or deletions.

In a further embodiment of the invention there is provided an isolated nucleic acid sequence comprising a sequence encoding the isolated polypeptide or fusion protein as described supra. Thus, a nucleic acid sequence encoding any of the isolated polypeptides or polypeptide fusion proteins described herein are also included as part of the instant invention. The skilled artisan will understand that a variety of nucleic acid sequences may be used to encode identical polypeptides in view of the degeneracy of genetic code. In certain cases for example the codon encoding any particular amino acid may be altered to improve cellular expression.

In preferred aspects, a nucleic acid sequence encoding the isolated polypeptide is comprised in an expression cassette. As used herein the term “expression cassette” means that additional nucleic acids sequences are included that enable expression of the isolated polypeptide in a cell, or more particularly in a eukaryotic cell. Such additional sequences may, for examples, comprise a promoter, an enhancer, intron sequences (e.g., before after or within the isolated polypeptide-encoding region) or a polyadenylation signal sequence. The skilled artisan will recognize that sequences included in an expression cassette may be used to alter the expression characteristics of the isolated polypeptide. For instance, cell type specific, conditional or inducible promoter sequences may be used to restrict expression of the isolated polypeptide to selected cell types or growth conditions. Furthermore, in some instances promoters with enhanced activity in cancer cells or pro-inflammatory immune cells. Furthermore, it is contemplated that certain alterations may be made to the isolated polypeptide-encoding sequence in order to enhance expression from an expression cassette for example, as exemplified herein, the initiation codon of the coding sequence of the isolated polypeptide may be changed to ATG to facilitate efficient translation.

In still further aspects of the invention a coding sequence of the isolated polypeptide may be comprised in an expression vector such as a viral expression vector. Viral expression vectors for use according to the invention include but are not limited to adenovirus, adeno-associated virus, herpes virus, SV-40, retrovirus and vaccinia virus vector systems. In certain preferred aspects, a retroviral vector may be further defined as a lentiviral vector. In some cases such lentiviral vectors may be self-inactivating (SIN) lentiviral vector such as those described in U.S. Applns. 20030008374 and 20030082789, incorporated herein by reference.

An isolated polypeptide of the embodiments may, in some aspects, bind to gp96 and inhibit its activity in a cell, specially a cancer cell or an inflammatory cell. There may be provided a pharmaceutical composition comprising the isolated polypeptide and a pharmaceutically acceptable carrier. In some respects, the invention provides methods for inhibiting or reducing gp96 activity comprising expressing the isolated polypeptide in a cell.

Thus, in a specific embodiment, there is provided a method for treating a subject with cancer or an inflammatory disease comprising administering to the subject an effective amount of a therapeutic composition comprising the isolated polypeptide or a nucleic acid expression vector encoding the isolated polypeptide as described supra. In a related aspect, there is provided a method of inhibiting cancer cell growth, cancer metastasis or inflammation in a subject, comprising administering an effective amount of the isolated polypeptide of the embodiments. In preferred aspects, methods described herein may be used to treat a human subject.

As described above, in certain aspects, the invention provides methods for treating cancer. In certain cases, the methods herein may be used to inhibit or treat metastatic cancers. A variety of cancer types may be treated with methods of the invention, for example a cancer for treatment may be a bladder, blood, bone, bone marrow, brain, breast, colon, esophagus, eye, gastrointestinal, gum, head, kidney, liver, lung, nasopharynx, neck, ovary, prostate, skin, stomach, testis, tongue, or uterus cancer. Furthermore additional anticancer therapies may be used in combination or in conjunction with methods of the invention. Such additional therapies may be administered before, after or concomitantly with methods of the invention. For example an additional anticancer therapy may be a chemotherapy, surgical therapy, an immunotherapy or a radiation therapy. In other aspects, the invention provides methods for treating inflammatory diseases such as sepsis, autoimmune disease, graft versus host diseases and graft rejection.

It is contemplated that compositions of the invention may be administered to a patient locally or systemically. For example, methods of the invention may involve administering a composition topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, intraperitoneally, subcutaneously, subconjunctival, intravesicularlly, mucosally, intrapericardially, intraumbilically, intraocularally, orally, by inhalation, by injection, by infusion, by continuous infusion, by localized perfusion bathing target cells directly, via a catheter, or via a lavage.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Integrin αL-132 interaction is gp96-dependent. (A) RAW264.7 cells were transduced with either empty vector (EV) or gp96 shRNA (1(D), and then levels of endogenous αL and β2 were immunoblotted. Surface expression of αL and β2 was analyzed by flow cytometry. (B) HA-tagged integrin αL and myc-tagged β2 were overexpressed in EV-transduced wild type (EV) and gp96 knock down (KD-1, KD-2) RAW264.7 cells. IP of HA-tagged integrin αL from EV and gp96 KD cells was done, followed by immunoblot (IB) for indicated proteins. Whole cell lysate (WCL) were used as control. Iso indicated IP with isotype control antibody. (C) IP of myc-tagged integrin β2 from gp96 EV and KD (KD-1) cells, followed by IB for indicated proteins. Whole cell lysate (WCL) were used as control. (D) Total lysates of HA-tagged αL-overexpressed EV-transduced and KD-1 RAW 264.7 cells were untreated, or treated with Endo H or PNGase F, followed by IB for integrin αL using anti-HA antibody. (E) EV and KD-1 cells were untreated, or treated with 5 μg/ml Tunicamycin for 12 hours, followed by IP for indicated proteins. WCL were used as control. (F) HA-tagged αL-overexpressed EV-transduced WT and KD-1 RAW264.7 cells were pulse labeled with [³⁵S] Met, followed by chasing with cold Met for indicated time point, and IP for αL-HA. The precipitated proteins were analyzed by SDS-PAGE and autoradiography.

FIG. 2. αI domain is critical for αL integrin to interact with gp96. (A) AID binds to gp96 in vitro. Murine B cell lysates were incubated with GST or GST-AID, recovered by glutathione-Sepharose 4B, and then resolved by SDS-PAGE. The associated gp96 and GST-AID were detected by IB. Equal amount of lysate were used as indicated by β-actin immunoblot. (B) WT αL-HA or AID deletion mutant (ΔAID) were transiently transfected into HEK293T cells. αL precipitates (IP:HA) were resolved by SDS-PAGE and immunoblotted for indicated proteins. The expression level of αL-HA and ΔAID mutant in the WCL were shown. (C) α7 helix is the critical region of AID to bind to gp96. Sequential deletion mutants of AID were fused with GST. GST pull-down assay was carried out. GST-AID deletion mutants and gp96 were detected by IB. FL: full length integrin αL.

FIG. 3. Overexpression of AID results in reduced surface expression of multiple integrins and cell invasion. (A) Confirmation of expression of FLAG-AID in RAW 264.7 macrophages by immunoblot. (B) Reduced surface expression of multiple gp96 clients (black-lined histogram) by flow cytometry. Gray-lined histograms represent isotype controls. Number represents mean fluorescence intensity (MFI) of integrin or TLR stain as indicated. (C) Invasion potential of EV-transduced or AID-overexpressing RAW 264.7 leukemia cells through an 8 μm diameter Transwell membrane after 15 hours of incubation. *P<0.03

FIG. 4. α7 helix peptide blocked interaction between gp96 and αL, and surface expression of multiple integrins. (A) IP of gp96 was carried out after 10 μM TAT-α7 helix peptide treatment for 12 hours, followed by IB for gp96 and αL-HA. Expression levels of indicated proteins in WCL were verified. β-actin is shown as a loading control. (B) PreB cells were treated with PBS or 10 μM TAT-α7 helix peptide for 12 hours, and then surface expression of integrin αL, αM, α4 and β1 was measured by flow cytometry. Number represents mean fluorescence intensity (MFI) of integrin stain. (C) CD44-stimulated αL expression was inhibited by cell permeable α7 helix peptide. HCT116 cells were pre-treated with 10 μM TAT-α7 peptide for 12 hours, and then incubated with control 2nd antibody or CD44 cross-link antibody for additional 12 hours. Cells were harvested, and flow cytometry was carried out for cell surface integrins. Histograms are a follows: IgG control and Non-cross link histograms appear as overlaid in the left panel, CD44 cross link (the histogram shifted to the right in left panel), CD44 cross link+TAT-α7 peptide (center histogram of the left panel).

FIG. 5. α7 helix peptide blocked cell invasion. (A) PreB leukemia cells were treated with the indicated concentrations of TAT-α7 helix peptide. MTT assay was carried out. (B) PreB and RAW264.7 cells were pre-treated with PBS or 10 μM TAT-α7 helix peptide for 12 hours, and then were incubated in a Transwell chamber for additional 15 hours to measure cell invasion. *P<0.05. (C) RPMI8226 myeloma cells were treated with PBS, 10 μM TAT-α7 helix peptide, 5 μM H39 or TAT-α7 plus H39 for 12 hours, and then the Transwell assay was performed. *P<0.05. (D) HCT116 cells were pre-treated with 10 μM TAT-α7 peptide for 12 hours, and then seeded into a Transwell chamber and incubated with control 2^{nd} antibody or CD44 antibody with/without 12 hour-pretreatment of TAT-α7 peptide for 12 hours. The numbers of invaded cells were counted. *P<0.05.

FIG. 6. A deletion mutant of the C-terminal loop structure abolishes the chaperone function of gp96. (A) Left, a WT gp96 homodimer structure is shown with the proposed CBD of gp96 (652-678) in light highlighted. The blow-up shows the CBD as a helix-loop structure in the C-terminal of gp96. Right, ΔCBD mutant is modeled to preserve the overall structure of gp96. (B) ΔCBD and WT gp96 exhibit identical behavior on gel filtration chromatography. 5-8 mg of purified protein was injected for each run, and the elution was monitored by absorbance at 280 nm. The peak at 73 ml contains gp96 dimer (˜200 kDa). (C) ΔCBD and WT gp96 exhibit identical ATP hydrolysis rates. ATP hydrolysis was measured using the PiPer assay system, which monitors free phosphate. The protein concentration in the reaction was 5 μm, and was carried out at 37° C. for 100 min. (D) ΔCBD mutant can be stably expressed in the gp96-null E4.126 cells. gp96, ΔCBD mutant and CNPY3-Flag were introduced into E4.126 cells by MigR retrovirus. Expression level was determined by SDS-PAGE. Empty virus (EV) was used as a control. (E) Both gp96 and ΔCBD mutant are able to interact with CNPY3. CNPY3-Flag was immunoprecipitated followed by immunoblot (IB) for gp96 or CNPY3. (F) Intracellular staining of gp96 and surface expression of integrins and TLRs (solid line histogram) in gp96-null pre-B cells transduced with WT or ΔCBD mutant. Gray histograms are isotype control antibody stain. Number represents mean fluorescence intensity (MFI) of TLRs or integrins. (G) NFκB-GFP reporter activation (green histogram) of cells in E after overnight (16-18 h) stimulation with Pam3CSK4 (10 μg/ml), LPS (10 μg/ml), CpG (5 μm), or P/I, which contains PMA (100 ng/ml) and ionomycin (2 μg/ml). Gray histograms are GFP profile of unstimulated cells.

FIG. 7. Interaction between gp96 and αL-integrin can be inhibited by a cell-permeable CBD peptide. HEK-293T cells were co-transfected with mouse gp96 and αL-HA. TAT-CBD peptides were added into medium 24 h post-transfection, and incubated for additional 24 h. Cells were then harvested. αL-HA precipitates were resolved and immunoblotted for mouse gp96 by a C-terminal mouse-specific gp96 antibody. Whole cell lysate (WCL) were also blotted for respective proteins as a control.

### DESCRIPTION OF ILLUSTRATIVE EMBODIMENTS

Integrins play important roles in regulating a diverse array of cellular functions crucial to the initiation, progression and metastasis of tumors. Studies have shown that a majority of integrins are folded by the ER chaperone gp96. gp96 (also known as grp94, endoplasmin, and HSP90b1) is the ER-resident member of the Hsp90 family. Its expression is upregulated by metabolic stress or the unfolded protein response (UPR), which results from the accumulation of misfolded proteins in the ER (Yang et al., 2005; Eletto et al., 2010; Li et al., 2011). gp96 has been implicated in cancer biology and, clinically, gp96 expression correlates with advanced stage and poor survival in a variety of cancers. gp96 expression is also closely linked to cancer growth and metastasis in melanoma, breast, prostate, multiple myeloma, lung cancer and colon cancer (Zheng et al., 2008; Missotten et al., 2003; Hodorova et al., 2008; Wu et al., 2007; Shen et al., 2002; Shen et al., 2002; Heike et al., 2000; Obeng et al., 2006; Usmani et al., 2010). gp96 has also been found to confer decreased sensitivity to X-ray irradiation (Lin et al., 2011), and it is required for the canonical Wnt pathway (Liu et al., 2013). Previously, however, there where no know molecules that could be used to inhibit gp96 activity.

Herein, it is shown that the dimerization of integrin αL and β2 is highly dependent on gp96. The Alpha I domain (AID), a ligand binding domain shared by seven integrin alpha subunits, is demonstrated to be a critical region for integrin binding to gp96. Deletion of AID significantly reduced the interaction between integrin αL and gp96. On the other hand, overexpression of AID intracellularly decreased surface expression of gp96 clients (integrins and TLRs) and cancer cell invasion. The α7 helix region is crucial for AID binding to gp96. A cell-permeable α7 helix peptide competitively inhibited the interaction between gp96 and integrins, and blocked cell invasion. Thus, targeting the binding site of α7 helix of AID on gp96 is an attractive new strategy for treatment of cancer and prevention of metastasis.

### I. INTEGRIN-BASED THERAPIES

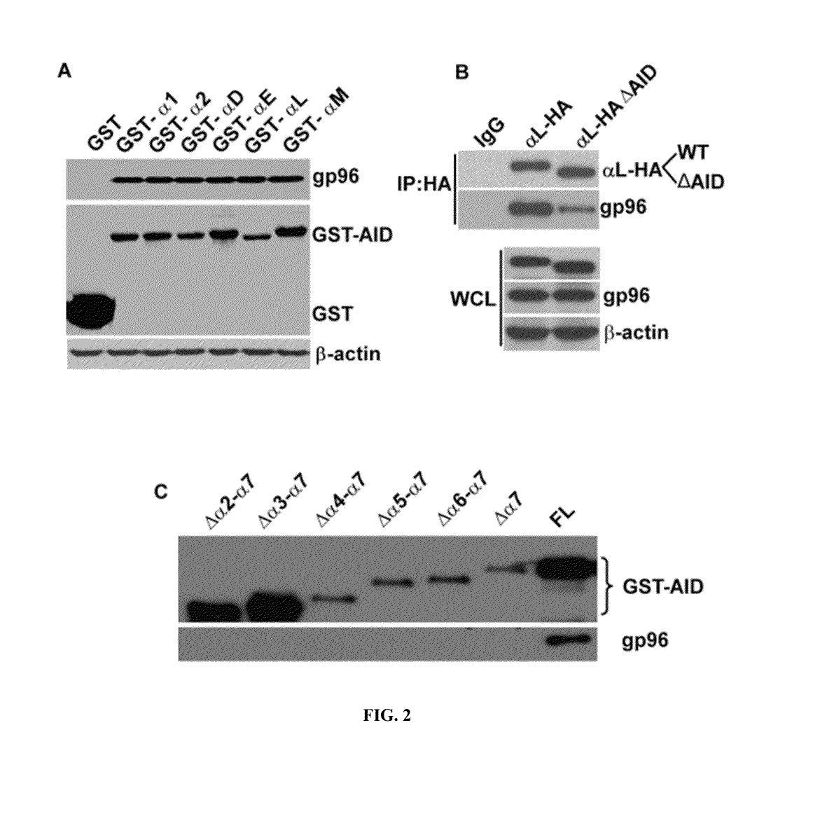

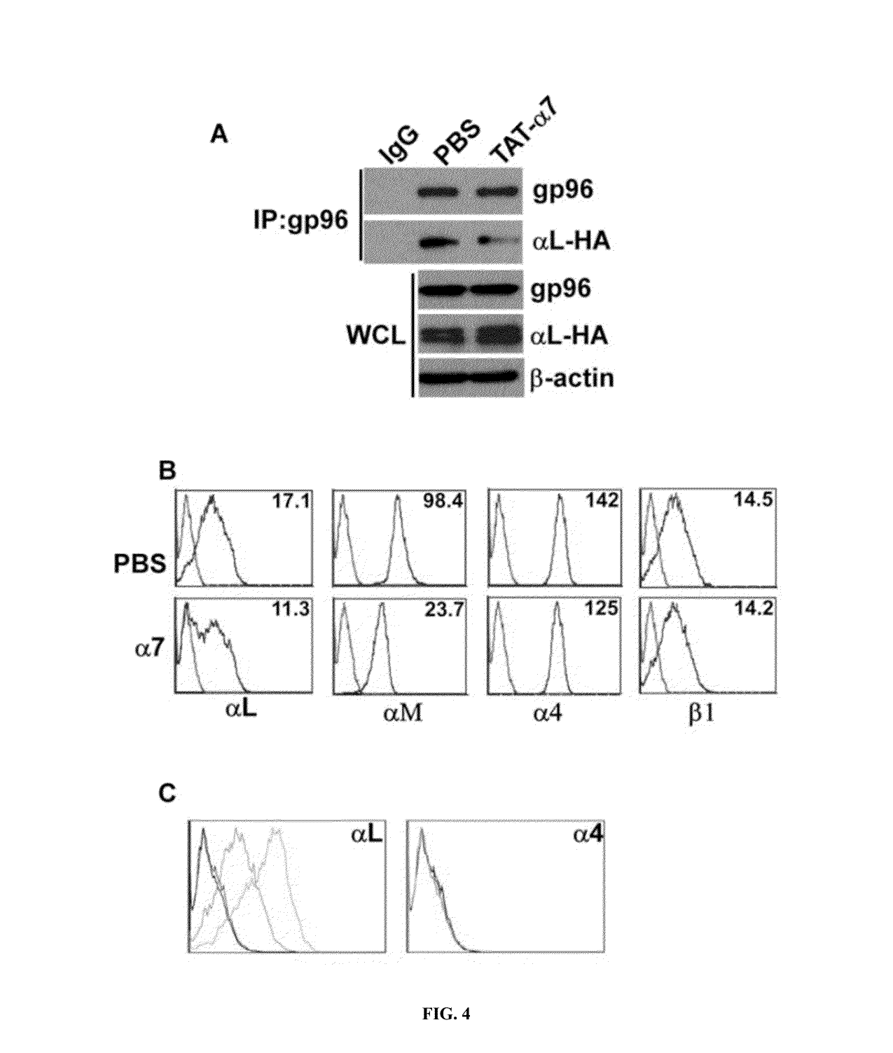

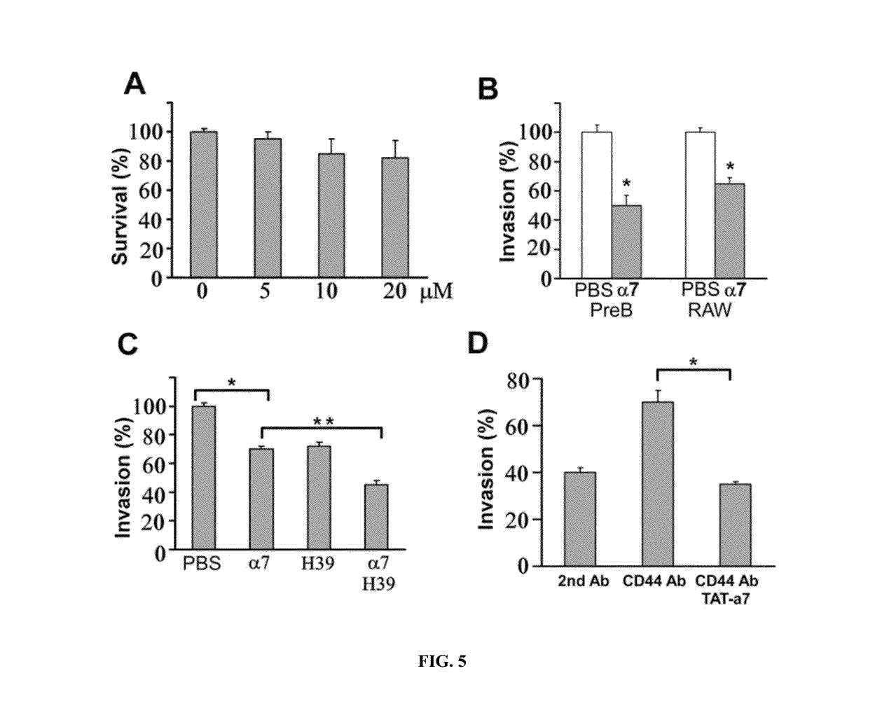

Many integrin-based inhibitors have thus far been introduced to the field for cancer therapy. However, these inhibitors only showed promising results in some preclinical studies, phase I/II clinical trials, but largely failed during clinical phase III trials (Bolli et al., 2009; Makrilia et al., 2009; Desgrosellier et al., 2010; Brooks et al., 1994; Gutheil et al., 2000; McNeel et al., 2005; Burkhart et al., 2004). The failure of these phase III trials can be ascribed to three causes: 1) Delivery. It is difficult to deliver the antibodies or peptides to tumors in humans even though preclinical studies show that the drugs have benefits in animal models; 2) Blocking Integrin blockade is incomplete due to dose, affinity, or accessibility problems; 3) Single target. Most of the inhibitors block the function of a single integrin, and it is possible that blocking multiple integrins could have better therapeutic effects. However, this approach has proven to be difficult, because most of the current integrin inhibitors are designed to compete with the ligands that bind to specific integrins. Such a strategy still allows for some ligand binding to other integrins that could trigger the outside-in signaling cascade in tumor cells. The studies disclosed herein are the first to show that AID is required for the interaction between integrin and gp96 (FIGS. 2A, B), and that the α7 helix of AID is critical for binding to gp96 (FIG. 2C). Of particular interest, gp96 plays a key role in the folding and cell surface expression of multiple integrin subunits, including α1, α2, α4, αD, αL, αM, αX, αV, αE, β2, β5, β6, β7, and β8 (Liu et al., 2008; Yang et al., 2007; Wu et al., 2012; Morales et al., 2009), many of which are critically required for tumor growth and metastasis (Albelda et al., 1990; Natali et al., 1997; Weaver et al., 2002; Guo et al., 2006; Dingemans et al., 2010; Fujisaki et al., 1999; Vincent et al., 1996; Matos et al., 2006). In this study, competitive blocking of the gp96-integrin interaction by TAT-α7 helix peptide decreased surface expression and maturation of not only integrin αL (see, e.g., NCBI accession no. NP_{—}001107852 (SEQ ID NO: 11), incorporated herein by reference), but also of other integrins (i.e., αM and α4) (FIGS. 4B, C). This allows targeting multiple integrins simultaneously, which is based on integrin substrate-derived peptide to occupy the client-binding site of gp96, to impair maturation of other gp96 clients. The residues 652-678 of client-binding domain (CBD) of gp96 are critical for its binding to both integrins and TLRs (Wu et al., 2012, incorporated by reference). Thus, the TAT-α7 helix peptide may bind and block the 652-678 region of the CBD. TAT-α7 helix peptide caused reduction of cell surface expression of multiple integrins (FIGS. 4B, 4C), as well as blocked cancer cell invasion in vitro (FIG. 5). Chaperone-based and client-specific inhibitors potentially hold a promise as a new class of therapeutics against cancer in the future.

### II. CELL INTERNALIZATION AND TARGETING MOIETIES

Cell internalization moieties or cell-targeting moieties for use herein may be any molecule in complex (covalently or non-covalently) with an isolated polypeptide described herein that mediates transport of the polypeptide across a cell membrane. Such internalization moieties may be polypeptides, polypeptides, hormones, growth factors, cytokines, aptamers or avimers. Furthermore, cell internalization moiety may mediate non-specific cell internalization or be a cell targeting moiety that is internalized in a subpopulation of targeted cells.

In certain aspects, polypeptides of the embodiments comprise or are conjugated to cell internalization moiety. As used herein the terms “cell internalization moiety” and “membrane translocation domain” are used interchangeably and refer to segments, e.g., of polypeptide sequence that allow a polypeptide to cross the cell membrane (e.g., the plasma membrane in the case of a eukaryotic cell). Examples of such segments include, but are not limited to, segments derived from HIV Tat, herpes virus VP22, the Drosophila Antennapedia homeobox gene product, or protegrin I.

In certain embodiments, cell targeting moieties for use in the current invention are antibodies. In general the term antibody includes, but is not limited to, polyclonal antibodies, monoclonal antibodies, single chain antibodies, humanized antibodies, minibodies, dibodies, tribodies as well as antibody fragments, such as Fab′, Fab, F(ab′)2, single domain antibodies and any mixture thereof. In some cases it is preferred that the cell targeting moiety is a single chain antibody (scFv). In a related embodiment, the cell targeting domain may be an avimer polypeptide. Therefore, in certain cases the cell targeting constructs of the invention are fusion proteins comprising an isolated polypeptide described herein and a scFv or an avimer. In some very specific embodiments the cell targeting construct is a fusion protein comprising an isolated polypeptide described herein fused to a single chain antibody.

In certain aspects, a cell targeting moieties may be a growth factor. For example, transforming growth factor, epidermal growth factor, insulin-like growth factor, fibroblast growth factor, B lymphocyte stimulator (BLyS), heregulin, platelet-derived growth factor, vascular endothelial growth factor (VEGF), or hypoxia inducible factor may be used as a cell targeting moiety according to the invention. These growth factors enable the targeting of constructs to cells that express the cognate growth factor receptors. For example, VEGF can be used to target cells that express FLK-1 and/or Flt-1. In still further aspects the cell targeting moiety may be a polypeptide BLyS (see U.S. Appln. 20060171919, incorporated by reference).

In further aspects of the invention, a cell targeting moiety may be a hormone. Some examples of hormones for use in the invention include, but are not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, erythropoitin, calcitriol, calciferol, atrial-natriuretic peptide, gastrin, secretin, cholecystokinin, neuropeptide Y, ghrelin, PYY3-36, insulin-like growth factor-1, leptin, thrombopoietin, angiotensinogen, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36. As discussed above targeting constructs that comprise a hormone enable method of targeting cell populations that comprise extracellular receptors for the indicated hormone. In yet further embodiments of the invention, cell targeting moieties may be cytokines, such as, granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-γ, IFN-α, IFN-β, LT-β, CD40 ligand, Fas ligand, CD27 ligand, CD30 ligand, 4-1BBL, TGF-β, IL 1α, IL-1 β, IL-1 RA, MIF and IGIF may all be used as targeting moieties according to the embodiments.

In certain aspects of the invention a cell targeting moiety of the invention may be a cancer cell targeting moiety. It is well known that certain types of cancer cells aberrantly express surface molecules that are unique as compared to surrounding tissue. Thus, cell targeting moieties that bind to these surface molecules enable the targeted delivery of an isolated polypeptide described herein specifically to the cancers cells. For example, a cell targeting moiety may bind to and be internalized by a lung, breast, brain, prostate, spleen, pancreatic, cervical, ovarian, head and neck, esophageal, liver, skin, kidney, leukemia, bone, testicular, colon or bladder cancer cell. The skilled artisan will understand that the effectiveness of cancer cell targeted polypeptide may, in some cases, be contingent upon the expression or expression level of a particular cancer marker on the cancer cell. Thus, in certain aspects there is provided a method for treating a cancer with targeted polypeptide comprising determining whether (or to what extent) the cancer cell expresses a particular cell surface marker and administering polypeptide therapeutic (or another anticancer therapy) to the cancer cells depending on the expression level of a marker gene or polypeptide.

### III. THERAPEUTIC COMPOSITIONS

Therapeutic compositions for use in methods of the invention may be formulated into a pharmacologically acceptable format. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one isolated polypeptide described herein or nucleic acid active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). A pharmaceutically acceptable carrier is preferably formulated for administration to a human, although in certain embodiments it may be desirable to use a pharmaceutically acceptable carrier that is formulated for administration to a non-human animal, such as a canine, but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an isolate polypeptide or its variant. In other embodiments, the polypeptide or its variant may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In particular embodiments, the compositions of the present invention are suitable for application to mammalian eyes. For example, the formulation may be a solution, a suspension, or a gel. In some embodiments, the composition is administered via a bioerodible implant, such as an intravitreal implant or an ocular insert, such as an ocular insert designed for placement against a conjunctival surface. In some embodiments, the therapeutic agent coats a medical device or implantable device.

Furthermore, the therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired. Thus, in some case dosages can be determined by measuring for example changes in serum insulin or glucose levels of a subject.

Precise amounts of the therapeutic composition may also depend on the judgment of the practitioner and are peculiar to each individual. Factors affecting the dose include the physical and clinical state of the patient, the route of administration, the intended goal of treatment (e.g., alleviation of symptoms versus attaining a particular serum insulin or glucose concentration) and the potency, stability and toxicity of the particular therapeutic substance.

For example, the composition may be a solution, a suspension, or a gel. In some embodiments, the composition is administered via a bioerodible implant, such as an intravitreal implant or an ocular insert, such as an ocular insert designed for placement against a conjunctival surface. In some embodiments, the therapeutic agent coats a medical device or implantable device.

In certain embodiments, therapeutic polypeptides or agents described herein may be operatively coupled to a targeting polypeptide or a second therapeutic agent, for example to form fusion or conjugated polypeptides. Agents or factors suitable for use may include any chemical compound that induces apoptosis, cell death, cell stasis and/or anti-angiogenesis. A second therapeutic agent may be a drug, a chemotherapeutic agent, a radioisotope, a pro-apoptosis agent, an anti-angiogenic agent, a hormone, a cytokine, a cytotoxic agent, a cytocidal agent, a cytostatic agent, a polypeptide, a protein, an antibiotic, an antibody, a Fab fragment of an antibody, a hormone antagonist, a nucleic acid or an antigen.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

Cell Lines.

All gp96 mutant-transduced PreB leukemia cell lines were generated from parental gp96-null E4.126 PreB cell line, which was a kind gift from Brian Seed (Harvard University). RAW 264.7 leukemia cell and HCT116 colon cancer cell lines were purchased from ATCC. Phoenix Eco (PE) packaging cell line from ATCC was used for retrovirus production. All culture conditions have been previously described (Liu et al., 2010).

Antibodies, Reagents and Peptides.

gp96 N terminus antibody 9G10 and gp96 C terminus antibody SPA851 were purchased from Enzo Life Sciences and detected both endogenous and overexpressed proteins. β-Actin antibody, Myc (9E10) and Flag antibody were from Sigma Aldrich. HA antibody (Clone 16B12) was purchased from Covance Inc. Biotin-conjugated anti-mouse CD11a (Clone: M174), CD49d (Clone: R1-2), CD18 (Clone: M18/2), TLR2 (Clone: 6C2), and TLR4 (Clone: MTS510) antibodies used for flow cytometry were purchased from eBioscience and they detected endogenous proteins. TAT-α7 peptide, containing TAT sequence (YGRKKRRQRRR; SEQ ID NO: 4) and amino acids 316-327 of integrin αL, was synthesized by NEO group to more than 98% purity as verified by HPLC and mass spectrometry. Other reagents were obtained from Sigma-Aldrich unless otherwise specified. H39, a gp96-specific purine scaffold inhibitor was synthesized as described previously (He et al., 2006).

Constructs and Site-directed Mutagenesis.

Wild-type murine integrin αL and β2 cDNA were used as templates for all PCR. Primers for integrin αL are 5′-ATTAGCGGCCGCGCCACCATGAGTTTCCGGATTGCGGG-3′ (SEQ ID NO: 7) and 5″-TAATGCGGCCGCTTAAGCATAATCTGGAACATCATATGGATAGTCCTTGTCACTCTC CCGGAGG-3′(SEQ ID NO: 8). Primers for integrin β2 are 5′-ATTAGCGGCCGCGCCACCATGCTGGGCCCACACTCACTG-3′ (SEQ ID NO: 9) and 5″-TAATGCGGCCGCCTACAGATCCTCTTCTGAGATGAGTTTTTGTTCGCTTTCAGCAAAC TTGGGGTTCATG-3′ (SEQ ID NO: 10). Integrin αL ΔAID were constructed by fusion PCR utilizing respective primers with Pfu (Invitrogen). All constructs were subcloned into MigR1 retroviral vector for retrovirus production as described previously (Liu et al., 2012).

Retrovirus Production and Transduction.

MigR1-integrin αL, β2 or AID plasmids were transfected into PE cell line using Lipofectamine 2000 (Invitrogen). Six hours after transfection, medium was replaced by pre-warmed fresh culture medium. Virus-containing medium was collected at 48 h after transfection. To facilitate the virus adhesion, spin transduction was performed at 1800×g for 1.5 h at 32° C. in the presence of 8 μg/ml hexadimethrine bromide (Sigma).

Blasticidin Selection.

A blasticidin resistant gene was bicistronically expressed downstream of the target gene in the MigR1 vector. All transduced PreB or RAW 264.7 cells were selected for a week in RPMI or DMEM culture medium containing 10 μg/ml blasticidin to ensure a relatively homogenous population and comparable expression level between all mutants.

Pulse-Chase Experiment.

HA-tagged integrin αL-overexpressing RAW 264.7 (WT and gp96 KD) cells were incubated with methionine- and cysteine-free medium for 2 hours, followed by pulsing with 110 μCi [³⁵S] methionine at 37° C. for 1 hour, and chased at 0, 1, 2 and 4 hours. Cells were washed with PBS, and lysed in PBS containing 5% SDS. Cells were freeze-thawed for 3 times to enhance lysis. 200 μg of lysate were immunoprecipitated using an anti-HA antibody, followed by SDS-PAGE and autoradiography.

Flow Cytometry.

All staining protocols, flow cytometry instrumentation as well as data analysis were performed as described previously without significant modifications (Yang et al., 2007; Liu et al., 2010; Staron et al., 2011). For cell surface staining, single cell suspension of living cells was obtained and washed with FACS buffer twice. FcR blocking with or without serum blocking was performed depending on individual primary antibody used for staining Primary and secondary antibodies staining were performed stepwise, with FACS buffer washing in between steps. Propidium iodide (PI) was used to gate out dead cells. Stained cells were acquired on a FACS Calibur or FACS verse (BD Biosciences) and analyzed using the FlowJo software (Tree Star).

GST Pull-Down Assay.

AID of mouse integrin and deletion mutants of α7 helix region of AID were subcloned into pGEX-pMagEmcs vector. GST fusion proteins were isolated on glutathione-Sepharose 4B beads (Amersham Biosciences). Cell lysate was incubated with GST alone or with GST-AID in the presence of 20 mM HEPES, pH 7.2, 50 mM KCl, 5 mM MgCl₂, 20 mM Na₂MO₄, 0.5% NP40, and 1 mM ATP, followed by incubation with glutathione-Sepharose 4B beads at 4° C. overnight, and then washed 3 times, boiled in Laemmli buffer, and resolved by SDS-PAGE.

Invasion Assay.

Cells (1×10⁵) were seeded in the upper chamber of a 1% gelatin-coated Transwell membrane (Corning). At 15 hours, cells were fixed in 90% ethanol for 10 minutes and stained with 1% crystal violet for 10 minutes. Cells in the lower chamber were eluted with 10% acetic acid for 10 minutes and cell number was determined by OD at 595 nm.

Statistical Analysis.

The Student t test was used for statistical analysis. P values <0.05 were considered significant.

### Example 2

### Alpha 7 Helix Region of Alpha I Domain (AID) is Crucial for Integrin Binding to ER Chaperone gp96

Formation of the Integrin Heterodimer is gp96-Dependent.

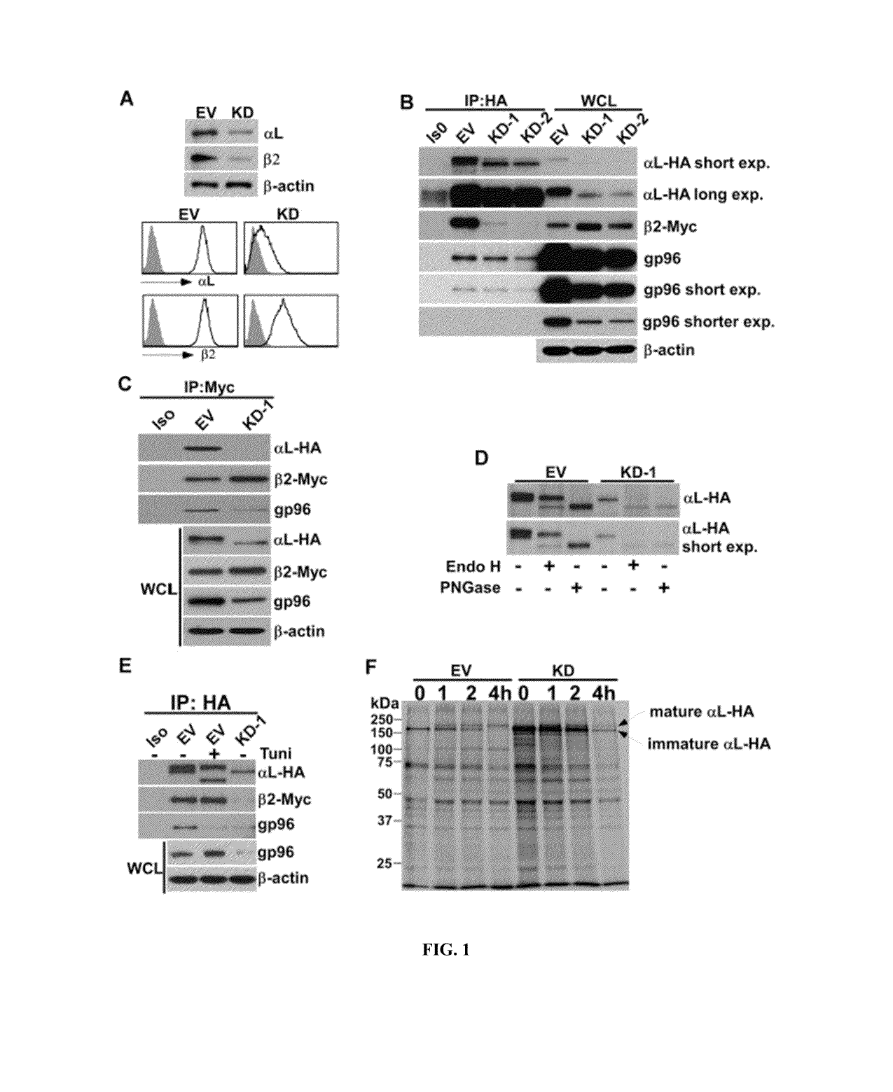

To test if gp96 is required for formation of the integrin heterodimer, the inventors used shRNA to knock down gp96 in RAW 264.7 macrophages. Both total and surface expression of αL and β2 were reduced in gp96 knockdown RAW 264.7 cells (1(D), comparing with that in wild-type cells transduced with empty vector (EV) (FIG. 1A). The inventors further overexpressed HA-tagged integrin αL and myc-tagged integrin β2 in EV-transduced WT or two KD RAW 264.7 leukemia cell lines (KD1 and KD2). The level of αL-HA in KD cells was much less than that in EV-transduced WT cells (FIG. 1B). The dimerization of αL-HA and β2-myc was also reduced dramatically in gp96 KD RAW 264.7 cells, compared to that in EV-transduced WT cells (FIG. 1B). Immunoprecipitation of β2-myc failed to pull down αL-HA in gp96 KD cells, indicating inefficient dimerization between integrin αL and β2 in gp96 KD cells (FIG. 1C). This suggests that gp96 is required for integrin αL binding to P2. Meanwhile, αL-HA presented as a doublet in both EV-transduced WT and KD RAW 264.7 cells (FIGS. 1B and D). The top band was the major form in EV-transduced WT cells, whereas, the lower band was dominant in KD RAW 264.7 cells. The top band was shown to be resistant to Endoglycosidase H (Endo H) treatment, suggesting that this is the matured cell surface form of αL-HA, while the lower band was sensitive to Endo H, indicating it as the immature ER form of αL-HA (FIG. 1D). Additionally, both bands were sensitive to peptide-N-glycosidase F (PNGase F), which cleaves the entire N-linked glycan. The immature ER αL-HA was also sensitive to Tunicamycin, an N-linked glycosylation inhibitor, causing reduction in binding to gp96 even though Tunicamycin induced gp96 upregulation via unfolded protein response (URP). However, the matured cell surface αL-HA was resistant to this blockade, and had no change in forming the dimerization with P2-myc (FIG. 1E). The inventor's previous study showed that less than 5% of gp96 was superglycosylated, and preferentially binds to its clientele such as Toll-like receptor 9 (TLR9). Massively increased gp96 upon Tunicamycin treatment was deglycosylated, and failed to interact with TLR9 (Yang et al., 2007). All these observation suggest that N-linked glycosylation on both gp96 and its clients are required for their optimal interaction. The inventors also performed the pulse-chase experiment to follow the newly synthesized αL-HA in gp96 KD cells. In EV-transduced WT cells, the mature αL-HA started to appear 1 hour after chasing, and had completely changed to the mature form 4 hours later. However, in gp96 KD cells (KD), the level of αL-HA was dramatically reduced after 4-hour chasing, and a majority of αL-HA remained immature (FIG. 1F).

AID is Crucial for the Interaction Between Integrins and gp96.

To determine if AID is required for AID-containing integrin binding to gp96, the inventors generated GST-tagged AID proteins from six AID-contained integrins including α1, α2, αD, αE, αL and αM subunits. All six GST-tagged AID proteins bound to gp96 (FIG. 2A). Moreover, when AID was deleted from integrin αL, the deletion resulted in significantly reduced interaction between integrin αL and gp96 (FIG. 2B). These results suggested that AID is a major binding region for integrin association with gp96. To further define which region of AID is critical for binding gp96, sequential deletion mutants of AID were generated. α7 helix is composed of 12 amino acids. Deletion of this region (Δα7) resulted in failure of AID to bind to gp96, indicating that α7 is integral to the binding of AID to gp96 (FIG. 2C).

AID Overexpression Decreased Cell Invasion In Vitro.

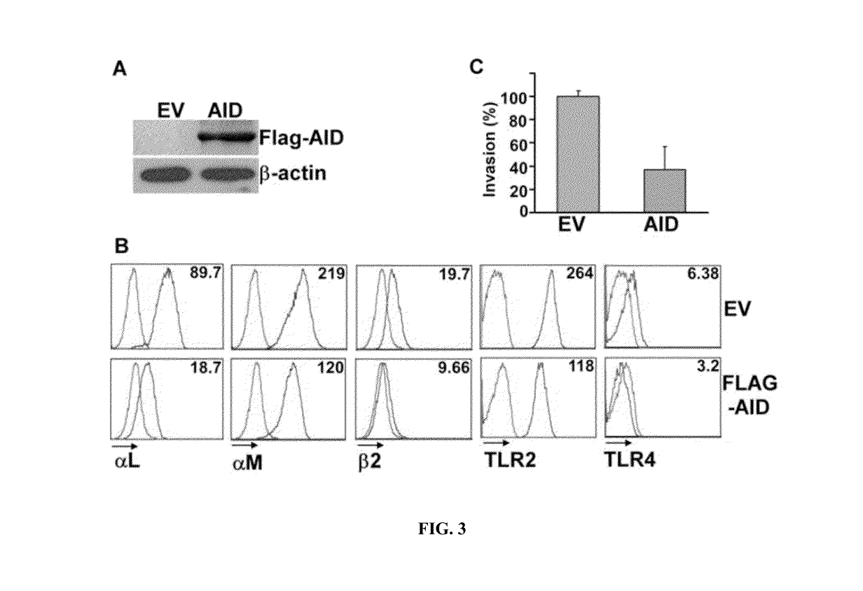

If AID is needed for integrin binding to gp96, then intracellular expression of isolated AID mini-protein in the ER should competitively bind to gp96, thereby reducing gp96 binding and surface expression of multiple endogenous clienteles. To test this hypothesis, the inventors overexpressed FLAG-tagged AID in RAW 264.7 cells by retroviral-mediated transduction (FIG. 3A), and found that surface expression of integrin αL, along with αM, β2, TLR2 and TLR4, was indeed decreased (FIG. 3B). In addition, AID-overexpressing cells also showed decreased cell invasion in a Transwell system (FIG. 3C).

Alpha 7 Helix Region of Alpha I Domain (AID) Interacts with the Client-Binding Domain (CBD) of ER Chaperone gp96.

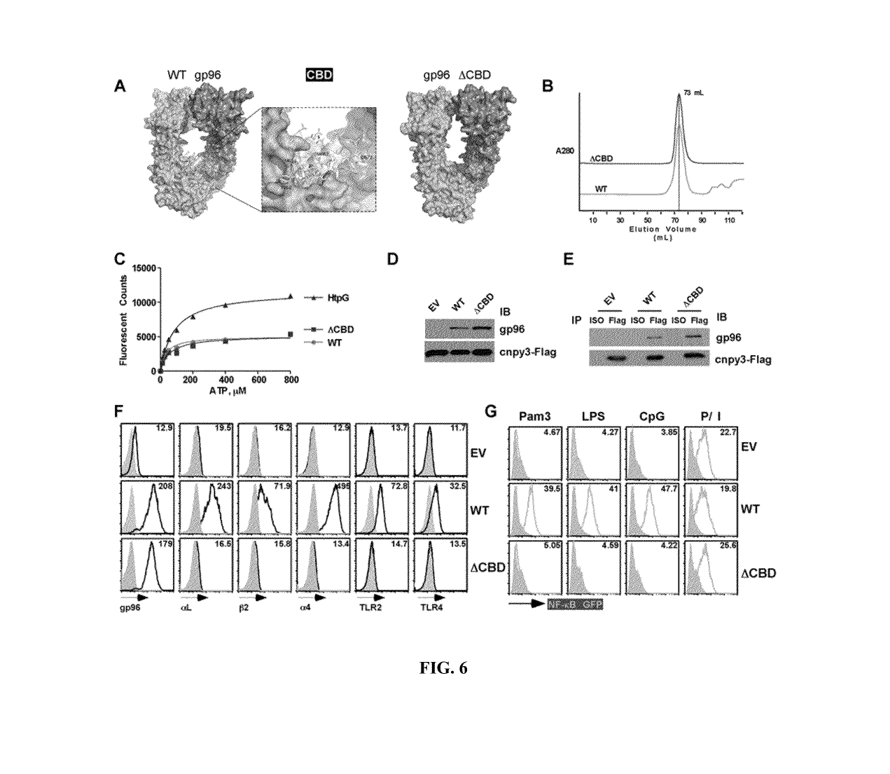

Genetic and biochemical evidence demonstrate that a C-terminal loop structure formed by residues 652-678, is the critical region of the client-binding domain (CBD) for both TLRs and integrins26 (FIG. 6A). Deletion of this region (ΔCBD) did not negatively affect the dimerization of gp96 (FIG. 6B), the intrinsic ATPase activity (FIG. 6C), the stable expression of the protein (FIG. 6D), or the ability of gp96 to interact with the TLR-specific co-chaperone CNPY4 (FIG. 6E). However, without it, the chaperoning function of gp96 collapsed (FIGS. 6F and 6G). While WT gp96 restored the surface expression of integrins and TLRs (FIG. 6F), ΔCBD was unable to rescue the expression of either of these clients. In addition, WT gp96 transduced cells responded well to stimulation by all TLR ligands tested, as measured by a NF-κB-GFP reporter assay. However, ΔCBD transduced cells failed to respond to any of the TLR ligands despite a similar reporter expression level as demonstrated by PMA/ionomycin stimulation (FIG. 6G).

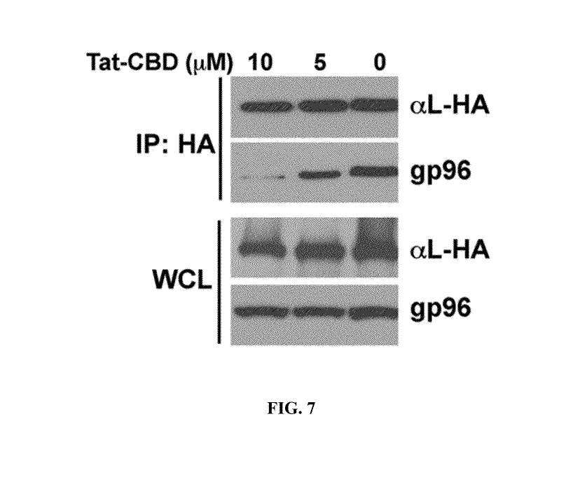

The possibility of direct binding between the CBD of gp96 and integrins was examined. A competition experiment was performed with a synthetic peptide that corresponds to CBD. Cells were incubated with increasing concentrations of a cell-permeable TAT-CBD peptide 24 h prior to cell lysis. IP analysis was performed to examine the interaction between gp96 and HA-tagged αL integrin. TAT-CBD inhibited the ability of gp96 to interact with αL-HA in a dose-dependent manner (FIG. 7). This supports there being a direct interaction between the CBD and αL integrin.

### Example 3

### Cell-Permeable α7 Helix Peptide is Effective Against Cancer Metastasis

Cell-permeable TAT-α7 peptide blocked interaction between gp96 and integrin αL. Since the α7 helix region is critical for AID binding to gp96, we synthesized a cell-permeable TAT-tagged α7 helix peptide to test whether or not it competes with the endogenous integrin αL. TAT is an HIV protein that plays a pivotal role in both the HIV-1 replication cycle and in the pathogenesis of HIV-1 infection. An HIV TAT-derived peptide enables the intracellular delivery of cargos of various sizes and physicochemical properties, including small particles, proteins, peptides, and nucleic acids (Zhao et al., 2004). The inventors performed a competition experiment by incubating cells with this TAT-α7 peptide for 24 h prior to cell lysis. The inventors then performed IP analysis to examine the interaction between gp96 and HA-tagged αL integrin. TAT-α7 peptide inhibited the ability of gp96 to interact with αL-HA (FIG. 4A). This further supports the suggestion that there is a direct interaction between gp96 and the AID of αL integrin through the α7 helix region. Furthermore, TAT-α7 peptide partially blocked surface expression of integrin αL, αM and α4, but not β1 (FIG. 4B).

CD44 cross-linking on cancer cells has been shown to increase the cell surface expression of integrin αL, resulting in increased cancer invasion (Fujisaki et al., 1999). To determine if the α7 helix peptide reduces CD44 cross-linking induced surface expression of integrin αL, the inventors treated the human colon cancer cell line, HCT116, with 10 μM TAT-tagged α7 helix peptide. Such a treatment resulted in complete abrogation of CD44-stimulated surface upregulation of αL (FIG. 4C).

TAT-α7 Helix Peptide Prevented Cell Invasion In Vitro.

Next, the inventors tested if TAT-α7 helix peptide can inhibit cell survival and invasion. As shown in FIG. 5A, a PreB leukemia cell line was treated with the indicated doses of TAT-α7 helix peptide, which had little effect on cell survival. However, when PreB and Raw 264.7 cells were pre-treated with 10 μM of TAT-α7 helix peptide, and then incubated in a Transwell system, cell invasion showed significant compromise, compared to PBS-treated cells (FIG. 5B). This reduced invasion was also observed in CD44 antibody-treated HCT116 cells with a pretreatment of the TAT-α7 helix peptide (FIG. 5D). The inventors also tested if this novel peptide inhibitor could potentiate the anti-tumor effect of H39, a highly selective gp96-specific inhibitor of the purine scaffold class (Taldone et al., 2009). H39 inhibits gp96 by directly binding to the ATP-binding pocket, but not the client-binding domain of gp96. TAT-α7 helix peptide and gp96-specific inhibitor, H39, had at least an additive effect on preventing invasion of RPMI8226 human myeloma cells (FIG. 5C).

Development of a Cell-Permeable α7 Helix Peptide for Treatment of Cancer in Vivo.

To overcome the generally unfavorable bioavailability of peptides in vivo, the peptide will be modified by forming a nano-complex with a zwitterionic polymer, or adding a free thiol group to the peptides, and then linking to the polymer through disulfide bonds, which will intracellularly release the peptide to form a cancer-targeted nanoparticle. This technology has been verified by using melittin, a 26 amino acid amphiphilic peptide isolated from honeybee (Apis mellifera) venom, as a model peptide (Soman et al., 2009). The single secured nano-sting (SSNS) was fabricated by mixing succinic anhydride modified glycol chitosan (SA-GCS) with melittin. Fluorescent measurement showed that with the increase of SA-GCS polymer, the detectable free melittin gradually decreases and achieved 100% encapsulation at a polymer to melittin ratio of 40. To further stabilize the complex, inhibit its premature release of melittin, and eliminate any potential side effects, SA-GCS was substituted with the SC-GCS—SH and the complexes were aerially oxidized to promote the formation of a disulfide bond among the SA-GCS—SH polymers to achieve dual secured nano-sting (DSNS). The formation of DSNS was confirmed by dynamic light scattering. The hydrodynamic size of DSNS was about 285 nm. The surface charge of the complexes at pH 7.4 was slightly negative, which is ideal for taking advantage of the enhanced permeability and retention effect (EPR) of cancer cells.

To confirm that the encapsulated peptide still retains its anticancer activity, MTT assays against HCT-116 human colon cancer cells will be performed. It is expected that free peptide, as well as peptide-packed SSNS and DSNS, will show dose-dependent cytotoxicity and kill almost 100% of cancer cells at μM concentrations. With melittin, the SSNS and DSNS nanoparticles were more effective in killing HCT-116 cells than free melittin. DSNS killed 100% of HCT-116 cells at the melittin concentration of 5 μM, at which free melittin could only partially kill cancer cells.

The α7 Helix Peptide Decreases Cancer Cell Migration and Attachment In Vitro.

The α7 helix peptide, and any derivatives identified through mutational analysis, will be tested to determine the most effective peptides for functional analysis and eventual in vivo testing. To confirm that the α7 helix peptide can block the maturation of integrins and cancer cell migration in other cell lines, TAT-tagged α7 helix peptide or control peptides at various concentrations (0, 2, 4, 6, 8 or 10 μM) will be delivered into multiple cell types, including RAW and PreB leukemia cells, MDA-MB231 breast cancer cells and HCT116 colon cancer cells. Transwell migration and scratch assays (Larrea et al., 2009) will be carried out for all the four cell lines to determine if the α7 helix peptide inhibits cell migration in vitro. The α7 helix peptide is expected to prevent surface expression of integrins and migration since all these cell lines express multiple integrins that are required for motility of these cancer cells.

Next, whether the α7 helix peptide blocks cell attachment will be tested. Various cancer cell lines will be pre-treated with control or TAT-α7 helix peptide (10 μM) for 1, 2 or 3 days, followed by seeding on ICAM-1-coated 96-well plates (1×10⁴ cells/well). After 30 min, non-adhering cells will be washed off, and attached cells counted at 200× magnification. An MTT solution in 10% FBS-containing medium will then be added, and ninety minutes later the absorbance at 570 nm will be recorded to indirectly quantify the density of adhering cells.

To improve the anti-tumor activity of the α7 helix peptide, it will be determined whether the α7 helix peptide has synergistic activity with other integrin inhibitors to block cell migration and induce cell death, such as LFA878, gp96 CBD peptide, or gp96 inhibitor (WS13 or H39). For migration assays, 1×10⁵ RAW, MDA-MB231 or HCT116 cells will be plated into a transwell chamber and treated with the following inhibitors or combinations of inhibitors for 12-24 hours: (i) 10 μM control peptide or TAT-α7 helix peptide alone; (ii) 10 μM LFA878 alone, 10 μM WS13 or H39 alone; (iii) 10 μM TAT-CBD peptide alone; (iv) 10 μM TAT-α7 helix peptide plus 10 μM WS13 or H39; (v) 10 μM TAT-α7 helix peptide+10 μM LFA878; or (vi) 10 μM TAT-α7 helix peptide+10 μM TAT-CBD peptide. The percentage of migrated cells over the total number of cells will be computed. For the apoptosis assay, tumor cells will be treated with these inhibitors for 24 hours at 60% confluence in 10% FBS-containing medium. Floating and attached cells will be resuspended in minimal essential medium containing 10% FBS, stained with 50 μg/ml propidium iodide (Sigma) and Annexin V-FITC (BioLegend), and analyzed by flow cytometry.

The α7 Helix Peptide Reduces Cancer Metastasis In Vivo.

Two reliable liver metastasis models of human colon cancer and mouse leukemia will be developed using immunodeficient NOD/scid IL2Rynull (NSG) (Jackson laboratory) mice and B6/DBA F1 mice, respectively. The HCT116 human colon cancer cell line highly expresses CD44 (Chen et al., 2011). Activation of CD44 by hyaluronan induces surface expression of integrin αL and augments LFA-1-mediated adhesion of cancer cells to endothelial cells (Fujisaki et al., 1999). Thus, the liver metastasis model will be performed with HCT116 cells. The PreB leukemia cell line 14.GFP is another line that widely disseminates upon injection due to the high level of integrins on its surface (Hewson et al., 1996). The activity of cell-permeable α7 helix peptide will be tested, alone or combination with other integrin inhibitors, in these models. In brief, 10⁴ HCT116 or PreB cells will be intrasplenetically injected into 7-8 week-old male NSG mice or B6/DBA F1 mice. One week later, mice will be divided into 4 groups (n=10/group), and treated with, (i) control peptide; (ii) TAT-CBD peptide; (iii) TAT-α7 helix peptide; (iv) TAT-α7 helix peptide+TAT-CBD. Control peptide and TAT-tagged α7 helix peptides will be injected intraperitoneally (3 mg/kg) once every two days for 4 weeks. NSG mice will receive one dose (1 mg/kg) of hyaluronan (Sigma) one day prior to peptide injection and then 1 mg/kg once every two days with peptide injection together. The mice will be sacrificed at 6 weeks after tumor cell injection. Liver metastatic nodules will be counted immediately using a surgical microscope, without fixation. Mice will be followed closely every week for body weight and signs and symptoms of distal organ dysfunction. Distressed mice will be humanely euthanized and a necropsy will be performed.

To increase the bioavailability of peptides and improve tumor targeting, the novel in vivo peptide delivery strategies described above will be applied. All peptides including TAT-α7 helix will be formed into a nano-complex with a zwitterionic polymer. The polymer complexes will be further linked by disulfide bonding to form the dual secured nano-particles. The effect of these nanopolymer-peptide complexes on cell migration and death will be evaluated in vitro by the standard MTT assay and cell migration assay before administration to mice. The polymers used will be biocompatible, and can protect the CBD and α7 helix peptides from degradation by peptidase/proteolysis through their “stealth” effect to achieve long circulation times and exhibit enhanced anticancer efficacy. Nanoparticles will target to the tumor sites through leaky blood capillaries and the lymphatic deficiency in the tumor tissue by a so-called enhanced permeability and retention (EPR) effect (Maeda et al., 2000; Fang et al., 2010; Fang et al., 2003). It has been demonstrated that by taking advantage of the EPR effect, nanoparticles can preferentially deliver drugs to cancer tissues, and therefore significantly enhance the therapeutic efficacy while substantially reducing drug side effects (Davis et al., 2008; Everts, 2007; Blanco et al., 2009).

In addition to the methods outlined above, an alternative protection method, which is to thiolate peptides, and then conjugate them to a novel nanogel system through a thiol-disulfide exchange reaction, may be employed. This nanogel system is based on polyethylene glycol modified poly[(2-(pyridin-2-yldisulfanyl)ethyl acrylate]. The system has been validated using the cRGD peptide, an integrin inhibitor, and camptothecin (CPT), a natural anti-cancer drug that inhibits DNA enzyme topoisomerase, as model compounds. The cRGD-SH peptide can be proportionally conjugated to the PDA-PEG copolymer. Thiolated CPT (CPT-SH) was also conjugated to PDA-PEG polymer through the same method. Dynamic light scattering demonstrated that nanogel fabricated from this technology has a size of around 100 nm. The release kinetics experiment indicates that the encapsulated drug is very stable inside the nanoparticle (pre-mature release free) while quickly releasing the drug in the environment with elevated redox potential (e.g., intracellular conditions).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Appl. No. 20030082789
U.S. Appl. No. 20030008374
U.S. Appl. No. 20060171919
U.S. Appl. No. 20060223114.
U.S. Appl. No. 20060234299
Albelda, S. M., Mette, S. A., Elder, D. E., Stewart, R., Damjanovich, L., Herlyn, M., and Buck, C. A. (1990) Cancer research 50, 6757-6764
Barczyk, M., Carracedo, S., and Gullberg, D. (2010) Cell and tissue research 339, 269-280
Blanco, E., Kessinger, C. W., Sumer, B. D. & Gao, J. Multifunctional Micellar Nanomedicine for Cancer Therapy. Exp. Biol. Med. 234, 123-131 (2009).
Bolli, N., De Marco, M. F., Martelli, M. P., Bigerna, B., Pucciarini, A., Rossi, R., Mannucci, R., Manes, N., Pettirossi, V., Pileri, S. A., Nicoletti, I., and Falini, B. (2009) Leukemia: official journal of the Leukemia Society of America, Leukemia Research Fund, U.K 23, 501-509
Brooks, P. C., Montgomery, A. M., Rosenfeld, M., Reisfeld, R. A., Hu, T., Klier, G., and Cheresh, D. A. (1994) Cell 79, 1157-1164
Burkhart, D. J., Kalet, B. T., Coleman, M. P., Post, G. C., and Koch, T. H. (2004) Molecular cancer therapeutics 3, 1593-1604
Chen, K. L. et al. Highly enriched CD133(+)CD44(+) stem-like cells with CD133(+)CD44(high) metastatic subset in HCT116 colon cancer cells. Clin Exp Metastasis 28, 751-63 (2011).
Cox et al., Integrins as therapeutic targets: lessons and opportunities. Nat Rev Drug Discov. 9(10):804-20, 2010
Davis, M. E., Chen, Z. G. & Shin, D. M. Nanoparticle therapeutics: an emerging treatment modality for cancer. Nat Rev Drug Discov 7, 771-82 (2008).
Desgrosellier, J. S., and Cheresh, D. A. (2010) Nature reviews. Cancer 10, 9-22
Dingemans, A. M., van den Boogaart, V., Vosse, B. A., van Suylen, R. J., Griffioen, A. W., and Thijssen, V. L. (2010) Molecular cancer 9, 152
Eletto, D., Dersh, D., and Argon, Y. (2010) Semin Cell Dev Biol 21, 479-485
Everts, M. Thermal scalpel to target cancer. Expert Rev Med Devices 4, 131-6 (2007).
Fang, J., Sawa, T. & Maeda, H. Factors and mechanism of “EPR” effect and the enhanced antitumor effects of macromolecular drugs including SMANCS. Adv Exp Med Biol 519, 29-49 (2003).
Fang, J., Nakamura, H. & Maeda, H. The EPR effect: Unique features of tumor blood vessels for drug delivery, factors involved, and limitations and augmentation of the effect. Adv Drug Deliv Rev (2010).
Fujisaki, T. et al. CD44 stimulation induces integrin-mediated adhesion of colon cancer cell lines to endothelial cells by up-regulation of integrins and c-Met and activation of integrins. Cancer Res 59, 4427-34 (1999).
Guo, W., Pylayeva, Y., Pepe, A., Yoshioka, T., Muller, W. J., Inghirami, G., and Giancotti, F. G. (2006) Cell 126, 489-502
Gutheil, J. C., Campbell, T. N., Pierce, P. R., Watkins, J. D., Huse, W. D., Bodkin, D. J., and Cheresh, D. A. (2000) Clinical cancer research: an official journal of the American Association for Cancer Research 6, 3056-3061
He, H., Zatorska, D., Kim, J., Aguirre, J., Llauger, L., She, Y., Wu, N., Immormino, R. M., Gewirth, D. T., and Chiosis, G. (2006) J Med Chem 49, 381-390
Heike, M., Frenzel, C., Meier, D., and Galle, P. R. (2000) International journal of cancer. Journal international du cancer 86, 489-493
Hewson, J., Bianchi, A., Bradstock, K., Makrynikola, V. & Gottlieb, D. Ultrastructural changes during adhesion and migration of pre-B lymphoid leukaemia cells within bone marrow stroma. Br J Haematol 92, 77-87 (1996).
Hodorova, I., Rybarova, S., Solar, P., Vecanova, J., Prokopcakova, L., Bohus, P., Solarova, Z., Mellova, Y., and Schmidtova, K. (2008) Neoplasma 55, 31-35
Larrea, M. D. et al. RSK1 drives p27Kip1 phosphorylation at T198 to promote RhoA inhibition and increase cell motility. Proc Natl Acad Sci USA 106, 9268-73 (2009).
Li, X., Zhang, K., and Li, Z. (2011) J Hematol Oncol 4, 8
Lin, C. Y., Lin, T. Y., Wang, H. M., Huang, S. F., Fan, K. H., Liao, C. T., Chen, I. H., Lee, L. Y., Li, Y. L., Chen, Y. J., Cheng, A. J., and Chang, J. T. (2011) Radiat Oncol 6, 136
Liu, B., and Li, Z. (2008) Blood 112, 1223-1230
Liu, B., Yang, Y., Qiu, Z., Staron, M., Hong, F., Li, Y., Wu, S., Hao, B., Bona, R., Han, D., and Li, Z. (2010) Nat Commun 1, doi:10 1038/ncommsl070
Liu, B., Staron, M., and Li, Z. (2012) PLoS One 7, e39442
Liu, B., Staron, M., Hong, F., Wu, B. X., Sun, S., Morales, C., Crosson, C. E., Tomlinson, S., Kim, I., Wu, D., and Li, Z. (2013) Proc Natl Acad Sci USA
Maeda, H., Wu, J., Sawa, T., Matsumura, Y. & Hori, K. Tumor vascular permeability and the EPR effect in macromolecular therapeutics: a review. J Control Release 65, 271-84 (2000).
Makrilia, N., Kollias, A., Manolopoulos, L., and Syrigos, K. (2009) Cancer investigation 27, 1023-1037
Matos, D. M., Rizzatti, E. G., Garcia, A. B., Gallo, D. A., and Falcao, R. P. (2006) Brazilian journal of medical and biological research=Revista brasileira de pesquisas medicas e biologicas/Sociedade Brasileira de Biofisica . . . [et al.] 39, 1349-1355
McNeel, D. G., Eickhoff, J., Lee, F. T., King, D. M., Alberti, D., Thomas, J. P., Friedl, A., Kolesar, J., Marnocha, R., Volkman, J., Zhang, J., Hammershaimb, L., Zwiebel, J. A., and Wilding, G. (2005) Clinical cancer research: an official journal of the American Association for Cancer Research 11, 7851-7860
Missotten, G. S., Journee-de Korver, J. G., de Wolff-Rouendaal, D., Keunen, J. E., Schlingemann, R. O., and Jager, M. J. (2003) Investigative ophthalmology & visual science 44, 3059-3065
Morales, C., Wu, S., Yang, Y., Hao, B., and Li, Z. (2009) J Immunol 183, 5121-5128
Natali, P. G., Hamby, C. V., Felding-Habermann, B., Liang, B., Nicotra, M. R., Di Filippo, F., Giannarelli, D., Temponi, M., and Ferrone, S. (1997) Cancer research 57, 1554-1560
Obeng, E. A., Carlson, L. M., Gutman, D. M., Harrington, W. J., Jr., Lee, K. P., and Boise, L. H. (2006) Blood 107, 4907-4916
Pontier, S. M., and Muller, W. J. (2009) Journal of cell science 122, 207-214
Raguse, J. D., Gath, H. J., Bier, J., Riess, H., and Oettle, H. (2004) Oral oncology 40, 228-230
Shen, C., Hui, Z., Wang, D., Jiang, G., Wang, J., and Zhang, G. (2002) Lung Cancer 38, 235-241
Soman, N. R. et al. Molecularly targeted nanocarriers deliver the cytolytic peptide melittin specifically to tumor cells in mice, reducing tumor growth. J Clin Invest 119, 2830-42 (2009).
Staron, M., Yang, Y., Liu, B., Li, J., Shen, Y., Zuniga-Pflucker, J. C., Aguila, H. L., Goldschneider, I., and Li, Z. (2010) Blood 115, 2380-2390
Staron, M., Wu, S., Feng, H., Stojanovic, A., Du, X., Bona, R., Liu, B., and Li, Z. (2011) Blood 117, 7136-7144
Taldone, T., and Chiosis, G. (2009) Current topics in medicinal chemistry 9, 1436-1446
Usmani, S. Z., Bona, R. D., Chiosis, G., and Li, Z. (2010) Journal of hematology & oncology 3, 40
Vanderslice et al., Integrin antagonists as therapeutics for inflammatory diseases. Expert Opin Investig Drugs. 15(10):1235-55, 2006
Vincent, A. M., Cawley, J. C., and Burthem, J. (1996) Blood 87, 4780-4788
Weaver, V. M., Lelievre, S., Lakins, J. N., Chrenek, M. A., Jones, J. C., Giancotti, F., Werb, Z., and Bissell, M. J. (2002) Cancer cell 2, 205-216
Wu, M., Bai, X., Xu, G., Wei, J., Zhu, T., Zhang, Y., Li, Q., Liu, P., Song, A., Zhao, L., Gang, C., Han, Z., Wang, S., Zhou, J., Lu, Y., and Ma, D. (2007) Proteomics 7, 1973-1983
Wu, S., Hong, F., Gewirth, D., Guo, B., Liu, B., and Li, Z. (2012) The Journal of biological chemistry 287, 6735-6742
Yang, Y., and Li, Z. (2005) Mol Cells 20, 173-182
Yang, Y., Liu, B., Dai, J., Srivastava, P. K., Zammit, D. J., Lefrancois, L., and Li, Z. (2007) Immunity 26, 215-226
Zhao, M., and Weissleder, R. (2004) Medicinal research reviews 24, 1-12
Zheng, H. C., Takahashi, H., Li, X. H., Hara, T., Masuda, S., Guan, Y. F., and Takano, Y. (2008) Human pathology 39, 1042-1049

What is claimed is:

1. An isolated polypeptide comprising the α7 helix peptide domain from integrin or a sequence having 1 or 2 amino acid substitutions, deletions or insertions relative to the α7 helix peptide domain, wherein the polypeptide is not a full length integrin polypeptide.

2. The isolated polypeptide of claim 1, wherein the α7 helix peptide domain from is from integrin αL.

3. The isolated polypeptide of claim 1, wherein the α7 helix peptide domain from is from human integrin αL (SEQ ID NO: 11).

4. The isolated polypeptide of claim 1, wherein the polypeptide is less than 200 amino acids in length.

5. The isolated polypeptide of claim 4, wherein the polypeptide is less than 50 amino acids in length.

6. The isolated polypeptide of claim 1, further conjugated to or fused with a cell-targeting or a cell internalization moiety.

7. The isolated polypeptide of claim 6, wherein the cell internalization moiety is at the N-terminus of the isolated polypeptide.

8. The isolated polypeptide of claim 6, wherein the cell internalization moiety is at the C-terminus of the isolated polypeptide.

9. The isolated polypeptide of claim 6, wherein the cell internalization moiety is a polypeptide, an aptamer, an antibody or an avimer.

10. The isolated polypeptide of claim 6, wherein the cell internalization moiety comprises internalization sequences selected from the group consisting of an HIV TAT protein transduction domain, HSV VP22 protein transduction domain, or Drosophila Antennapedia homeodomain.

11. The isolated polypeptide of claim 6, wherein the cell internalization moiety comprises a poly-arginine, a poly-methionine and/or a poly-glycine polypeptide.

12. The isolated polypeptide of claim 10, wherein the cell internalization moiety comprises the amino acid sequence GRKKRRQRRR (SEQ ID NO: 2), YGRKKRRQRRR (SEQ ID NO: 4) RMRRMRRMRR (SEQ ID NO: 5) or GRKKRRQRRRPQ (SEQ ID NO: 6).

13. The isolated polypeptide of claim 6, comprising the sequence at least 90% identical to SEQ ID NO: 3 (GRKKRRQRRRPQEKLKDLFTDLQR).

14. The isolated polypeptide of claim 1, wherein the α7 helix peptide domain comprises the sequence of SEQ ID NO: 1 or SEQ ID NO: 12 or a sequence having 1 or 2 amino acid substitutions, deletions or insertions relative to these sequences.

15. The isolated polypeptide of claim 9, wherein the antibody is an IgA, an IgM, an IgE, an IgG, a Fab, a F(ab′)2, a single chain antibody, or a paratope peptide.

16. An isolated nucleic acid comprising a nucleic acid segment encoding the isolated polypeptide of claim 1.

17. A pharmaceutical composition, comprising the polypeptide of claim 1 and a pharmaceutically acceptable carrier.

18. A method inhibiting cancer cell growth, cancer metastasis or inflammation in a subject, comprising administering an effective amount of the polypeptide of claim 1 to the subject.

19. The method of claim 18, wherein the subject has a cancer.

20. The method of claim 18, wherein the subject has an inflammatory disease.


SEQUENCE LISTING (13 sequences)

SEQ ID NO: 1 - protein, 12 aa, Mus musculus
EKLKDLFTDLQR

SEQ ID NO: 2 - protein, 10 aa, Human immunodeficiency virus
GRKKRRQRRR

SEQ ID NO: 3 - protein, 24 aa, Artificial Sequence
  Synthetic fusion protein
GRKKRRQRRRPQEKLKDLFTDLQR

SEQ ID NO: 4 - protein, 11 aa, Human immunodeficiency virus
YGRKKRRQRRR

SEQ ID NO: 5 - protein, 10 aa, Artificial Sequence
  Synthetic peptide
RMRRMRRMRR

SEQ ID NO: 6 - protein, 12 aa, Human immunodeficiency virus
GRKKRRQRRRPQ

SEQ ID NO: 7 - DNA, 38 bp, Artificial Sequence
  Synthetic primer
attagcggccgcgccaccatgagtttccggattgcggg

SEQ ID NO: 8 - DNA, 64 bp, Artificial Sequence
  Synthetic primer
taatgcggccgcttaagcataatctggaacatcatatggatagtccttgtcactctcccggagg

SEQ ID NO: 9 - DNA, 39 bp, Artificial Sequence
  Synthetic primer
attagcggccgcgccaccatgctgggcccacactcactg

SEQ ID NO: 10 - DNA, 70 bp, Artificial Sequence
  Synthetic primer
taatgcggccgcctacagatcctcttctgagatgagtttttgttcgctttcagcaaacttggggttcatg

SEQ ID NO: 11 - protein, 1086 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 12 - protein, 12 aa, Homo sapiens
EKLKDLFTELQK

SEQ ID NO: 13 - protein, 24 aa, Artificial Sequence
  Synthetic polypeptide
GRKKRRQRRRPQEKLKDLFTELQK